(19) United States
(12) Patent Application Publication
(10) Pub. No.: US 2023/0081356 A1
(43) Pub. Date: Mar. 16, 2023

(54) DELIVERY SYSTEM WITH DEVICE DEPLOYMENT FEATURES, AND ASSOCIATED METHODS

(71) Applicant: W. L. Gore & Associates, Inc., Newark, DE (US)

(72) Inventors: Robert M. Depue, Flagstaff, AZ (US); Benjamin Pohlman, Flagstaff, AZ (US); James D. Silverman, Flagstaff, AZ (US); Tyson J. Skelton, Flagstaff, AZ (US)

(21) Appl. No.: 17/917,293

(22) PCT Filed: Apr. 6, 2021
(86) PCT No.: PCT/US2021/025962

§ 371 Date: Oct. 6, 2022

Related U.S. Application Data
(60) Provisional application No. 63/005,722, filed on Apr. 6, 2020.

(51) Int. Cl. A61F 2/966

(52) U.S. Cl. CPC A61F 2/9662; A61F 2002/9665

(57) ABSTRACT

Delivery systems (100) are described that include endoprostheses (102) configured to transition from compact, delivery configurations to enlarged, deployed configurations, delivery catheters including body (108) and tip (112) portions, the endoprostheses being received on body portions in the compact, delivery configuration, the tip portions including lips (114) configured to define pockets (114A) that releasably receive the endoprosthesis in the compact, delivery configuration, and constraints releasably maintaining the endoprostheses in the compact, delivery configuration. Additional and alternative delivery system features and associated methods are provided.

### CROSS-REFERENCE TO RELATED APPLICATION

This application is a national phase application of PCT Application No. PCT/US2021/025962, internationally filed on Apr. 6, 2021, which claims the benefit of U.S. Application No. 63/005,722, filed Apr. 6, 2020, which are herein incorporated by reference in their entireties for all purposes.

### FIELD

The present disclosure generally relates to medical device delivery systems, and more particularly, to device deployment features of such systems.

### BACKGROUND

Various implantable devices are positioned and deployed at a treatment site in a body of a patient through endoluminal means, using a transcatheter approach. Such devices may be transitionable from a smaller profile, delivery configuration to an enlarged profile, deployed configuration. In some instances, the delivery systems employed to deliver and deploy such devices include removable and/or retractable sheaths or sleeves that are removed from the device to facilitate transitioning to the deployed configuration.

### SUMMARY

Delivery systems are described that include endoprostheses configured to transition from compact, delivery configurations to enlarged, deployed configurations, delivery catheters including body and tip portions, such endoprostheses being received on body portions in the compact, delivery configuration, the tip portions including lips configured to define pockets that releasably receive the endoprosthesis in the compact, delivery configuration, and constraints releasably maintaining the endoprostheses in the compact, delivery configuration. Additional and alternative delivery system features and associated methods are provided.

According to a first example (“Example 1”), a delivery system includes an endoprosthesis having a first end and a second end, the endoprosthesis configured to transition from a compact, delivery configuration to an enlarged, deployed configuration, a delivery catheter including a body portion and a tip portion, the endoprosthesis being received on the body portion of the delivery catheter in the compact, delivery configuration, the tip portion including a lip configured to define a pocket that releasably receives the first end of the endoprosthesis in the compact, delivery configuration, and a constraint releasably maintaining the endoprosthesis in the compact, delivery configuration, the constraint having a first end and a second end, the constraint extending over at least a portion of the endoprosthesis and the lip of the tip portion.

According to another example further to Example 1 (“Example 2”), the constraint includes a tubular, knitted sleeve.

According to another example further to Examples 1 or 2 (“Example 3”), the constraint is configured to unravel and deconstruct to release the endoprosthesis.

According to another example further to any preceding Example (“Example 4”), the lip of the tip portion is interposed between the first end of the endoprosthesis and the constraint.

According to another example further to any preceding Example (“Example 5”), the endoprosthesis includes a support portion formed of one or more rows defined by a stent element defining an undulating pattern, and further wherein the first end of the endoprosthesis releasably received in the pocket includes at least one of the one or more rows defined by the stent element.

According to another example further to any preceding Example (“Example 6”), the first end of the endoprosthesis is configured to release from the pocket as the endoprosthesis transitions from the compact, delivery configuration to the enlarged, deployed configuration.

According to another example further to any preceding Example (“Example 7”), the lip of the tip portion is elastically deformable.

According to another example further to any preceding Example (“Example 8”), the lip of the tip portion is configured to transition to from a first configuration to a second configuration following release of the endoprosthesis from the pocket defined by the lip, the second configuration of the lip having a lower profile than the first configuration of the lip.

According to another example further to any preceding Example (“Example 9”), the tip portion includes a serrated portion configured to mechanically engage the first end of the constraint.

According to another example (“Example 10”), a delivery catheter includes a body portion configured to receive an endoprosthesis in a compact, delivery configuration and a tip portion including a lip configured to releasably receive a first end of an endoprosthesis in a compact, delivery configuration.

According to another example further to Example 10 (“Example 11”), the lip of the tip portion is elastically deformable.

According to another example further to Examples 10 or 11 (“Example 12”), the lip of the tip portion is configured to elastically transition from a first configuration to a second configuration, the second configuration of the lip having a lower profile than the first configuration of the lip.

According to another example further to any one of Examples 10 to 12 (“Example 13”), the tip portion includes a serrated portion configured to mechanically engage a constraint for an endoprosthesis.

According to another example (“Example 14”), a delivery system includes an endoprosthesis having a first end and a second end, the endoprosthesis configured to transition from a compact, delivery configuration to an enlarged, deployed configuration and a delivery catheter including a body portion and a tip portion, the endoprosthesis being received on the body portion of the delivery catheter in the compact, delivery configuration, the tip portion including a lip configured to define a pocket that releasably receives the first end of the endoprosthesis in the com pact, delivery configuration, the lip being configured to elastically transition from a first configuration to a second configuration upon removal of the first end of the endoprosthesis from the pocket, the second configuration having a lower profile than the first configuration.

According to another example further to Example 14 (“Example 15”), the delivery system further comprises a constraint maintaining the endoprosthesis in the compact, delivery configuration.

According to another example further to Example 15 (“Example 16”), the constraint is configured to unravel and deconstruct to release the endoprosthesis.

According to another example further to Examples 15 or 16 (“Example 17”), the lip of the tip portion is interposed between the first end of the endoprosthesis and the constraint.

According to another example further to any one of Examples 14 to 17 (“Example 18”), the endoprosthesis includes a support portion formed of one or more rows defined by a stent element defining an undulating pattern, and further wherein the first end of the endoprosthesis releasably received in the pocket includes at least one of the one or more rows defined by the stent element.

According to another example further to any one of Examples 14 to 18 (“Example 19”), the first end of the endoprosthesis is configured to release from the pocket as the endoprosthesis transitions from the compact, delivery configuration to the enlarged, deployed configuration.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A illustrates a delivery system according to some embodiments.

FIG. 1B illustrates a selected portion of the delivery system corresponding to the area designated as B-B in FIG. 1A, according to some embodiments.

FIG. 2A illustrates a tip portion of a delivery system with an implantable device received by the tip portion, according to some embodiments.

FIG. 2B illustrates a tip portion of a delivery system, according to some embodiments.

FIG. 3A illustrates another tip portion of a delivery system with an implantable device received by the tip portion, according to some embodiments.

FIG. 3B illustrates another tip portion of a delivery system, according to some embodiments.

FIGS. 4A to 4C illustrate a deployment sequence, according to some embodiments.

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning those in the field would attribute such terminology.

Persons skilled in the art will readily appreciate that various aspects of the present disclosure can be realized by any number of methods and apparatuses configured to perform the intended functions. Stated differently, other methods and apparatuses can be incorporated herein to perform the intended functions. It should also be noted that the accompanying drawing figures referred to herein are not all drawn to scale, but may be exaggerated to illustrate various aspects of the present disclosure, and in that regard, the drawing figures should not be construed as limiting. Finally, although the present disclosure may be described in connection with various principles and beliefs, the present disclosure should not be bound by theory.

The term “distal” refers to a relative location that is farther from a location in the body at which the medical device was introduced. Similarly, the term “distally” refers to a direction away from a location in the body at which the medical device was introduced.

The term “proximal” refers to a relative location that is closer to the location in the body at which the medical device was introduced. Similarly, the term “proximally” refers to a direction towards a location in the body at which the medical device was introduced.

### Description of Various Embodiments

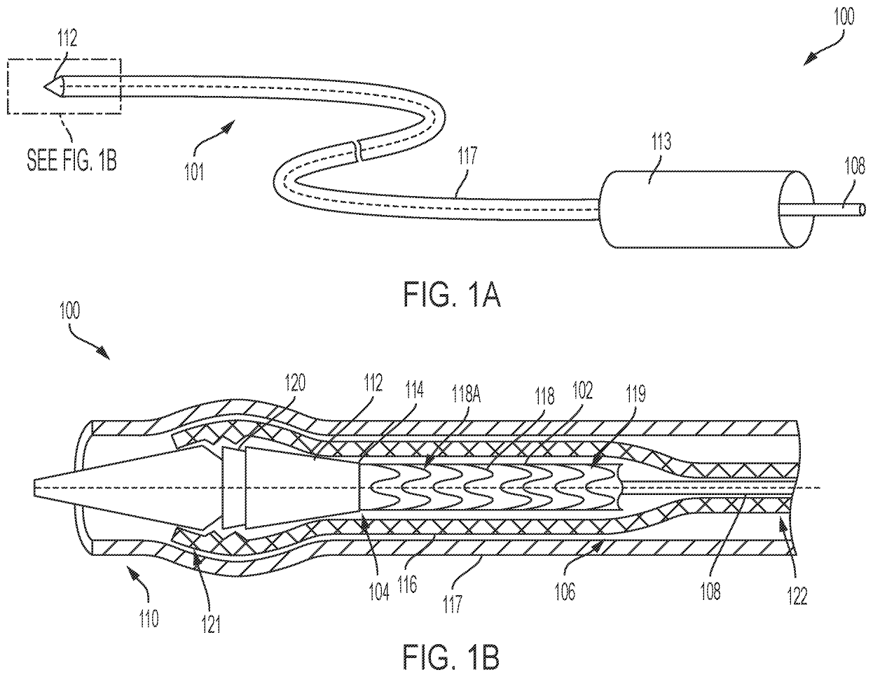

FIG. 1A shows a delivery system 100 including an endoprosthesis 102, a delivery catheter 101 including a body portion 108, a tip portion 112 coupled to the body portion 108, and a handle portion 113 coupled to the body portion 108, a constraint 116 with a first end 121 and a second end 122 (see FIG. 1B) extending along at least a portion of the body portion 108, the endoprosthesis 102, and the tip portion 112, and an outer sheath 117 configured to facilitate endoluminal passage of the delivery system 100 (e.g., with a relatively smooth outer profile), and in particular the endoprosthesis 102, to a treatment site in the body. Generally, the delivery system 100 is configured to position the endoprosthesis 102 at a desired treatment site in the body of a patient and to control deployment of the endoprosthesis 102 from a compact, delivery configuration to an expanded, deployed configuration.

In various examples, the endoprosthesis 102 is a device configured to replace or repair a body part, the endoprosthesis 102 being implantable (permanently or temporarily) inside of a body of a patient. In various examples, the endoprosthesis 102 includes a support portion 118 (e.g., a stent), and may include an optional covering 119 (e.g., graft material). Examples of the endoprosthesis 102 include stents, stent-grafts, grafts, filters, occluders, balloons, leads, prosthetic valves, and others. As shown, the endoprosthesis 102 has a first end 104 and a second end 106. As subsequently described, the first end 104 of the endoprosthesis 102 is releasably retained and/or covered by the tip portion 112 when the endoprosthesis 102 is in the compact delivery configuration.

The support portion 118 of the endoprosthesis 102 may include one or more stent elements extending in an undulating pattern that defines one or more rows 118A. The rows 118A may be defined by one or more turns of the stent elements (e.g., where the undulating pattern extends helically to define a tubular shape. Alternatively, the rows 118A may be configured as one or more discrete rings that collectively define a tubular shape. The support portion 118 may be formed by winding techniques (e.g., wire winding or braiding) or by forming techniques (e.g., cutting, depositing or otherwise forming a pattern in a tubular construct or a sheet that is subsequently formed into a tubular construct), for example. The support portion 118 may be formed from metallic materials, polymeric materials, natural materials, or combinations thereof as desired. The support portion 118 can comprise conventional medical grade materials such as nylon, polyacrylamide, polycarbonate, polyethylene, polyformaldehyde, polymethylmethacrylate, polypropylene, polytetrafluoroethylene, polytrifluorochloroethylene, polyvinylchloride, polyurethane, elastomeric organosilicon polymers; metals such as stainless steels, cobalt-chromium alloys and nitinol and biologically derived materials such as bovine arteries/veins, pericardium and collagen. The support portion 118 can also comprise bioresorbable materials such as poly(amino acids), poly(anhydrides), poly(caprolactones), poly(lactic/glycolic acid) polymers, poly(hydroxybutyrates) and poly(orthoesters).

The covering 119 of the endoprosthesis 102 may be configured as a graft, for example. Potential materials for the covering 119 include, for example, expanded polytetrafluoroethylene (ePTFE), polyester, polyurethane, fluoropolymers, such as perfluoroelastomers and the like, polytetrafluoroethylene, silicones, urethanes, ultra-high molecular weight polyethylene, aramid fibers, and combinations thereof. Other embodiments for a graft member material can include high strength polymer fibers such as ultra-high molecular weight polyethylene fibers or aramid fibers. Further, a graft may comprise a class of polyesters such as polyethylene terephthalate and polyaramids, polyfluorocarbons such as polytetrafluoroethylene (PTFE) with and without copolymerized hexafluoropropylene, and porous or nonporous polyurethanes.

The delivery catheter 101 may take on a variety of configurations, but is generally suited to position the endoprosthesis in the body of the patient (not shown) through an endoluminal approach. The body portion 108 of the delivery catheter 101 may be tubular and elongate in form and provides a means for maintaining and supporting the endoprosthesis as it traverses the body to the desired treatment site. The handle portion 113 of the delivery catheter 101 is configured to assist with manipulation of the delivery catheter 101 to facilitate passing the endoprosthesis 102 to the treatment site and may include deployment features (e.g., actuator(s) for causing the endoprosthesis 102 to expand), steering features (e.g., actuator(s) for steering the delivery catheter 101 through the body), or others.

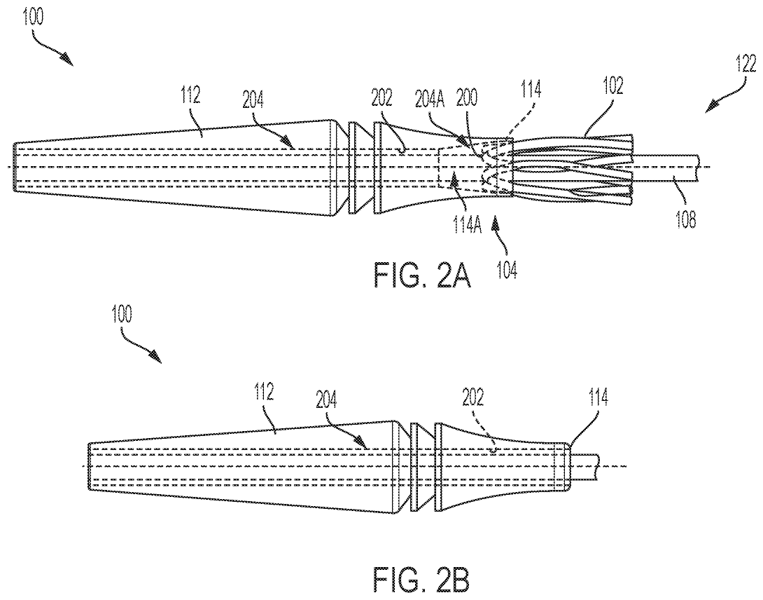

As shown, the tip portion 112 of the delivery catheter 101 extends from a distal end 110 of the body portion 108. In some examples, the tip portion 112 has a lip 114 interposed between the first end 104 of the endoprosthesis 102 and the constraint 116 configured to define a pocket 114A (see FIG. 2A) (e.g., in combination with the body portion 108) that releasably receives the first end 104 of the endoprosthesis 102 when the endoprosthesis 102 is in the compact delivery configuration. The first end 104 of the endoprosthesis 102 may include at least one of the rows 118A defined by the stent elements of the support portion 118. In some examples, the lip 114 is formed of an elastomeric material, or is otherwise configured to be elastically deformable. The first end 104 is released from the pocket 114A as the endoprosthesis 102 transitions from the compact, delivery configuration to the enlarged, deployed configuration. In some examples, the lip 114 is configured to deflect outwardly to form the pocket 114A and receive the first end 104 of the endoprosthesis 102 and to retract, or collapse to a smaller profile when the first end 104 is removed from the pocket 114A. By elastically recovering toward a smaller profile, the lip 114 helps retain the first end 104 of the endoprosthesis 102 and/or decreases the profile of the tip portion 112 following release of the endoprosthesis to ease retraction of the delivery catheter 101 from the body of the patient. The lip 114, in some embodiments, may provide a radially compressive force to at least partially retain the first end 104 of the endoprosthesis 102 in a compact, delivery configuration. Furthermore, the lip 114 and the first end 104 of the endoprosthesis 102 may be engaged such that a frictional force between the lip 114 and the endoprosthesis 102 resists longitudinal movement of the endoprosthesis 102 relative to the tip portion 112 when engaged. The constraint 116 may further increase the frictional force between the endoprosthesis 102 and the lip 114 when the constraint 116 is maintaining the endoprosthesis in the compact, delivery configuration. The tip portion 112 can be formed of a deformable material, such as a low durometer polymeric material, for example a biocompatible elastomer. In some examples, the tip portion 112 is formed of a low durometer polymeric material having a durometer between 15 and 70 Shore on the Type A scale.

In some embodiments, the tip portion 112 includes a serrated portion 120 including one or more (e.g., 2, 3, 4, 5, 6, or more) serrations. The serrated portion 120 may be configured to mechanically engage with the constraint 116 to help prevent the constraint 116 from slipping or translating (e.g., during passage through the body of a patient and/or during release of the constraint 116). For example, in embodiments where the constraint 116 is tensioned longitudinally in a proximal direction to release the constraint 116 from the endoprosthesis 102, it may be helpful to have features that assist with maintaining the position of the constraint 116 to avoid inadvertent slipping or other translation.

In some embodiments, the serrated portion 120 is portion is formed on the outer surface of the tip portion 112. For example, the outer surface of the tip portion may include a generally smooth outer profile except at the serrated portion 120. For example, the outer surface of the tip portion 112 may include transitions in the outer profile that are rounded or curved, or form an obtuse angle except at the serrated portion 120 which may include portions of the outer surface that form acute angles. With reference to FIG. 2A, for example, the serrated portion 120 includes an outer profile forming acute angles along the profile of the body, wherein the tips of the serrated portion 120 are operable to contact and maintain the constraint 116 at a predetermined longitudinal position. The serrated portion 120 may also extend circumferentially around the tip portion 112 at a predetermined longitudinal position along the length of the tip portion 112. In some embodiments, the serrated portion 120 may be described as having a plurality of frustoconical shaped portions positioned adjacent each other.

In some examples, the constraint 116 extends along the endoprosthesis 102 and over the lip 114 of the tip portion 112, as well as partially along the body portion 108 and partially along the tip portion 112. Generally, the constraint 116 surrounds at least a portion of the endoprosthesis 102 when the endoprosthesis 102 is in the compact delivery configuration. Where the endoprosthesis 102 is self-expanding, the constraint 116 may help keep the endoprosthesis 102 in the compact delivery configuration. In examples where the endoprosthesis is expandable (e.g., balloon expandable) the constraint may passively constrain the endoprosthesis 102 (e.g., not being required to prevent expansion, but in fact surrounding and covering the endoprosthesis 102) and act to primarily cover or protect the endoprosthesis 102, rather than to actively constrain, for example.

In various examples, the constraint 116 is configured as a tubular, knit sleeve of material that is releasable by unraveling and/or deconstructing upon tensioning a portion of the sleeve (e.g., a pull line). Such tubular knit sleeves may comprises one or more knitted fibers which can, for example, comprise a woven warp knit or knit-braid. Suitable examples of knit sleeves and associated deployment features and techniques may be found in U.S. Pat. No. 6,315,792 to Armstrong et al., issued Nov. 13, 2001, entitled “Remotely removable covering and support.” In various examples, one layer of the constraint 116 is configured to first unravel in a first direction (e.g., proximal to distal) and then a second layer (e.g., an underlying layer) is configured to unravel in a second direction (e.g., distal to proximal). In such a manner, the constraint can be configured to deploy an endoprosthesis “tip first,” where a first end (e.g., a distal end) of the endoprosthesis is first released from the constraint and then the remainder of the endoprosthesis is released in a proximal direction. In other examples, the constraint is configured as a tubular sheath of material with edges secured together with a coupling element (e.g., with a filament) that may then be released by tensioning the coupling element such that the tubular sheath opens. In still other examples, the constraint 116 is a tubular member that is retractable to release the endoprosthesis.

As shown, the endoprosthesis 102 is received over the body portion 108 in the compact delivery configuration such that the endoprosthesis 102 is positioned proximal toward a distal end 110 of the body portion 108. In operation, the endoprosthesis 102 is generally expandable to an enlarged deployed configuration and, in the case of various examples, the endoprosthesis 102 separates from the tip portion 112 following expansion of the endoprosthesis 102. As shown, the first end 104 of the endoprosthesis 102 is received in the pocket 114A and is revealed and retracts out from under the lip 114 as the endoprosthesis 102 expands towards the enlarged deployed configuration either as or after the constraint 116 is removed.

In some examples, one or more of the rows 118A of the support portion 118 forms the portion of the first end 104 of the endoprosthesis 102 that is received in the pocket 114A formed by the lip 114 of the tip portion 112. For example, the distal most of the rows 118A may form the portion of the first end 104 of the endoprosthesis 102 received in the pocket 114A. In some embodiments, the one or more rows 118A define a plurality of apices 200 that are received in the pocket 114A of the lip 114.

In various examples, the tip portion 112 defines an inner lumen 204 that passes longitudinally through the tip portion 112 (e.g., which assists with securing the body portion 108 to the tip portion 112, in various examples. In examples where the tip portion 112 is elastic, the inner lumen 204 is expandable, or distensible by deflecting the lip 114, such that the inner lumen 204 is transitionable between configurations corresponding to an expanded lumen 204A and a retracted lumen 204B. The expanded lumen 204 configuration corresponds to the condition when the lip 114 receives the first end 104 of the endoprosthesis 102 and is interposed between the constraint 116 and the first end 104 of the endoprosthesis 102. FIG. 2B shows the delivery system 100 where the endoprosthesis 102 (FIG. 2A) is removed from the tip portion 112 such that the first end 104 no longer comes into contact with the lip 114 and the tip portion 112 defines the retracted lumen 204B configuration. In the retracted lumen 204B configuration or state, the tip portion 112 returns to a smaller profile.

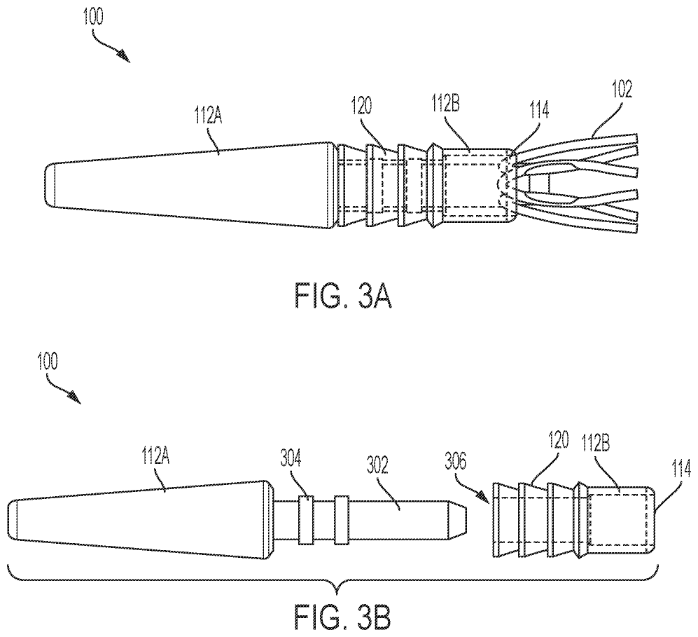

FIGS. 3A and 3B show the delivery system 100 according to various examples. In the illustration of FIGS. 3A and 3B, the body portion 108 is not shown for ease of visualizing other components of the system 100. As indicated, the tip portion 112 is optionally configured as a multi-component assembly, with a first component 112A coupled to a second component 112B. As shown, the first component 112A includes an insertable portion 302 with one or more retention features 304 and the second component 112B includes the serrated portion 120 and a bore 306 configured to receive the insertable portion 302. The insertable portion 302 inserts into the bore 306 with the lip 114 and the insertable portion 302 defining the pocket 114A for receiving the first end 104 of the endoprosthesis 102.

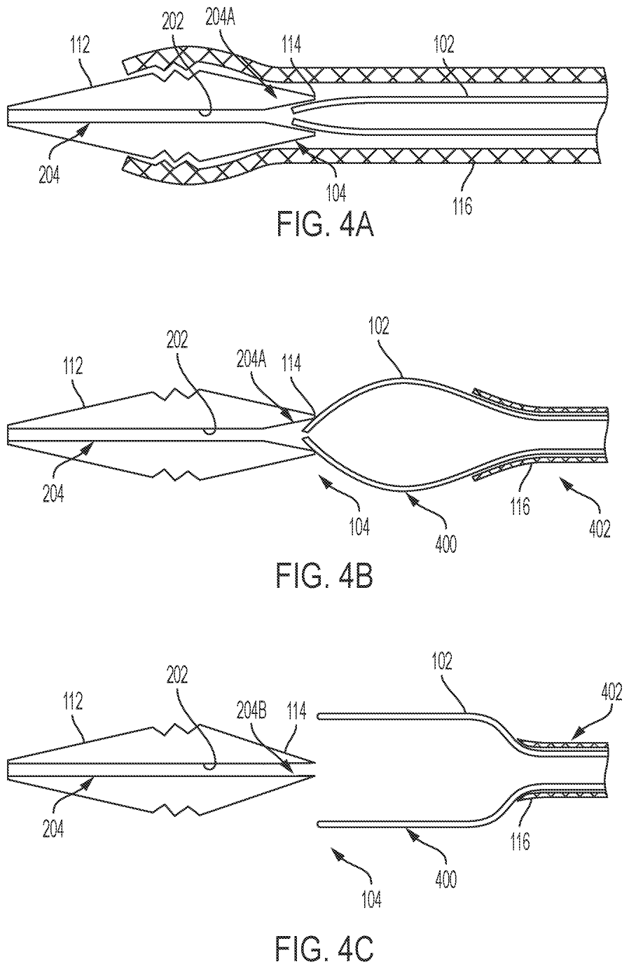

FIGS. 4A, 4B, and 4C illustrate a deployment process according to some examples, and is demonstrative of a manner in which the first end 104 of the endoprosthesis 102 separates from the tip portion 112 according to some embodiments. For simplicity, the body portion 108 is not shown in these figures. In FIG. 4A, the endoprosthesis 102 is constrained by both the lip 114 of the tip portion 112 and the constraint 116 to assume the compact delivery configuration with the lip 114 interposed between the first end 104 of the endoprosthesis 102 and the constraint 116. According to various examples, when receiving the first end 104 of the endoprosthesis 102, the lumen 204 within the tip portion 112 is biased, or expanded outwardly to accept the first end 104.

In FIG. 4B, the constraint 116 is partially pulled from the endoprosthesis 102. The lip 114 protects the first end 104 of the endoprosthesis 102, separating it from the constraint 116, which can help avoid snagging of the constraint 116 on the first end 104 and/or otherwise facilitates smooth release of the constraint 116 over the first end 104. As shown, the constraint 116 is removed from the tip portion 112, and a portion of the endoprosthesis proximal the first end 104, but still constrains a portion of the endoprosthesis 102 proximal the first end 104. The endoprosthesis 102 is shown constrained partially by the lip 114 of the tip portion 112. The unconstrained portion 400 of the endoprosthesis 102 expands outwardly (radially) toward the enlarged deployed configuration, while the first end 104 (constrained by the lip 114) is retained at least partially in a compact, delivery configuration. As shown, the first end 104 begins to angulate and/or retract from the pocket 114A as the portion of the endoprosthesis 102 adjacent the tip portion 112 expands.

In FIG. 4C, the constraint 116 is pulled further from the endoprosthesis 102 and the outward expansion force of the endoprosthesis 102 overcomes the engagement with the lip 114, causing the first end 104 to be released from the pocket 114A to expand outwardly and/or radially toward the enlarged deployed configuration. In some examples, a portion (e.g., a proximal portion) of the endoprosthesis 102 remains constrained by the constraint 116, while the first end 104 is free from the lip 114. As shown, the lumen 204 optionally retracts to a low profile configuration after release of the first end 104 of the endoprosthesis 102. The constraint 116 is then removed/released from the endoprosthesis as desired and the endoprosthesis is transitioned to the enlarged, deployed configuration.

A method of assembling a delivery system 100 for the endoprosthesis 102 is provided. The method includes positioning an endoprosthesis 102 having a first end 104 in a compact, delivery configuration on an elongate element or body portion 108, elastically deforming a lip 120 of a tip portion 120 to receive the first end 104 of the endoprosthesis 102, and positioning the tip portion 112 on the elongate element adjacent the first end 104 of the endoprosthesis 102, the first end 104 of the endoprosthesis 102 being releasably received within the lip 114 of the tip portion 112. The lip 120 may be elastically deformed such that the lip 120 expands radially outwardly and forms a pocket 114a or the pocket 114a is enlarged sufficiently to receive the first end 104 of the endoprosthesis 102. The method may also include placing a constraint 116 radially outward of or around the endoprosthesis 102. The constraint 116 may be positioned radially outward of the lip 114 of the tip portion 112. In some embodiments, the constraint 116 may also be positioned over a serrated portion 120, the serrated portion 120 operable to contact and maintain the constraint 116 and resist longitudinal movement of the constraint 116 relative to the tip portion 112, the endoprosthesis 102 and the body portion 108.

The embodiments have been described above both generically and with regard to specific embodiments. It will be apparent to those skilled in the art that various modifications and variations can be made in the embodiments without departing from the scope of the disclosure. Thus, it is intended that the embodiments cover the modifications and variations of the embodiments provided they come within the scope of the appended claims and their equivalents.

1. A delivery system comprising:
an endoprosthesis having a first end and a second end, the endoprosthesis configured to transition from a compact, delivery configuration to an enlarged, deployed configuration;
a delivery catheter including a body portion and a tip portion, the endoprosthesis being received on the body portion of the delivery catheter in the compact, delivery configuration, the tip portion including a lip configured to define a pocket that releasably receives the first end of the endoprosthesis in the compact, delivery configuration; and
a constraint releasably maintaining the endoprosthesis in the compact, delivery configuration, the constraint having a first end and a second end, the constraint extending over at least a portion of the endoprosthesis and the lip of the tip portion.

2. The delivery system of claim 1, wherein the constraint includes a tubular, knitted sleeve.

3. The delivery system of claim 1, wherein the constraint is configured to unravel and deconstruct to release the endoprosthesis.

4. The delivery system of claim 1, wherein the lip of the tip portion is interposed between the first end of the endoprosthesis and the constraint.

5. The delivery system of claim 1, wherein the endoprosthesis includes a support portion formed of one or more rows defined by a stent element defining an undulating pattern, and further wherein the first end of the endoprosthesis releasably received in the pocket includes at least one of the one or more rows defined by the stent element.

6. The delivery system of claim 1, wherein the first end of the endoprosthesis is configured to release from the pocket as the endoprosthesis transitions from the compact, delivery configuration to the enlarged, deployed configuration.

7. The delivery system of claim 1, wherein the lip of the tip portion is elastically deformable.

8. The delivery system of claim 1, wherein the lip of the tip portion is configured to transition to from a first configuration to a second configuration following release of the endoprosthesis from the pocket defined by the lip, the second configuration of the lip having a lower profile than the first configuration of the lip.

9. The delivery system of claim 1, wherein the tip portion includes a serrated portion configured to mechanically engage the first end of the constraint.

10. A delivery catheter comprising:
a body portion configured to receive an endoprosthesis in a compact, delivery configuration; and
a tip portion including a lip configured to releasably receive a first end of an endoprosthesis in a compact, delivery configuration.

11. The delivery catheter of claim 10, wherein the lip of the tip portion is elastically deformable.

12. The delivery catheter of claim 10, wherein the lip of the tip portion is configured to elastically transition from a first configuration to a second configuration, the second configuration of the lip having a lower profile than the first configuration of the lip.

13. The delivery catheter of claim 10, wherein the tip portion includes a serrated portion configured to mechanically engage a constraint for an endoprosthesis.

14. A delivery system comprising:
an endoprosthesis having a first end and a second end, the endoprosthesis configured to transition from a compact, delivery configuration to an enlarged, deployed configuration; and
a delivery catheter including a body portion and a tip portion, the endoprosthesis being received on the body portion of the delivery catheter in the compact, delivery configuration, the tip portion including a lip configured to define a pocket that releasably receives the first end of the endoprosthesis in the compact, delivery configuration, the lip being configured to elastically transition from a first configuration to a second configuration upon removal of the first end of the endoprosthesis from the pocket, the second configuration having a lower profile than the first configuration.

15. The delivery system of claim 14, further comprising a constraint maintaining the endoprosthesis in the compact, delivery configuration.

16. The delivery system of claim 15, wherein the constraint is configured to unravel and deconstruct to release the endoprosthesis.

17. The delivery system of claim 15, wherein the lip of the tip portion is interposed between the first end of the endoprosthesis and the constraint.

18. The delivery system of claim 14, wherein the endoprosthesis includes a support portion formed of one or more rows defined by a stent element defining an undulating pattern, and further wherein the first end of the endoprosthesis releasably received in the pocket includes at least one of the one or more rows defined by the stent element.

19. The delivery system of claim 14, wherein the first end of the endoprosthesis is configured to release from the pocket as the endoprosthesis transitions from the compact, delivery configuration to the enlarged, deployed configuration.

20. A method of assembling an endoprosthesis delivery device comprising:
positioning an endoprosthesis having a first end in a compact, delivery configuration on an elongate element;
elastically deform ing a lip of a tip portion to receive the first end of the endoprosthesis; and
positioning the tip portion on the elongate element adjacent the first end of the endoprosthesis, the first end of the endoprosthesis being releasably received within a lip of the tip portion.
